(19) United States
(12) Patent Application Publication
(10) Pub. No.: US 2024/0174972 A1
(43) Pub. Date: May 30, 2024

(54) PROCESS FOR THE RECOVERY OF MICROALGAE USING MAGNETIC NANOPARTICLES OF BACTERIAL ORIGIN

(71) Applicants: PETRÓLEO BRASILEIRO S.A. - PETROBRAS, Rio de Janeiro (BR); UNIVERSIDADE FEDERAL DO RIO DE JANEIRO, Rio de Janeiro (BR)

(72) Inventors: LEONARDO BRANTES BACELLAR MENDES, RIO DE JANEIRO (BR); RAFAEL RICHARD JOAO, RIO DE JANEIRO (BR); IGOR NUNES TAVEIRA, RIO DE JANEIRO (BR); TARCISIO NASCIMENTO CORREA, RIO DE JANEIRO (BR); JÚLIA CUNHA DE CASTRO, RIO DE JANEIRO (BR); FERNANDA DE AVILA ABREU, RIO DE JANEIRO (BR); RONALDO BERNARDO DA SILVA, RIO DE JANEIRO (BR); ROGERIO PRESCILIANO DE SOUZA FILHO, RIO DE JANEIRO (BR)

(21) Appl. No.: 18/315,169

(22) Filed: May 10, 2023

(30) Foreign Application Priority Data
Nov. 29, 2022 (BR) 10 2022 024357 3

(51) Int. Cl. C12N 1/12; C12N 1/02

(52) U.S. Cl. CPC C12N 1/12; C12N 1/02

(57) ABSTRACT

The invention is part of methods for separating microorganisms from their culture media and aims at solving the low efficiency and high environmental impact of processes for recovering oil-producing microalgae. For this purpose, the invention provides for the application of magnetic nanoparticles of bacterial origin (NMOBs) in processes for isolating microalgae from a cell culture or from a suspension of salt water or an effluent. NMOBs are particularly capable of recovering microalgae from hypersaline suspensions, favoring the use of marine microalgae in the production of oils with potential application in bio-oil and biofuel production processes. In addition to the use of NMOBs, the invention also provides for a process for recovering microalgae based on the addition of NMOBs to the microalgae suspension and subsequent application of external magnetic force.

### FIELD OF THE INVENTION

The invention pertains to methods of separating microorganisms from their culture media and has particular application to the concentration and purification of microalgae. The described method aims at providing a more cost-effective way to supply oleaginous microalgae biomass for biofuel production processes.

### BACKGROUND OF THE INVENTION

In recent years, greater emphasis has been given to microalgae due to its possible use as a sustainable substitute for oil derivatives and fuel products. The technology for producing advanced biofuels and other bioproducts from microalgae fits into the context of the transition to a low-carbon economy, already chosen as a national strategy by highly developed countries such as Sweden and Switzerland.

However, one of the biggest challenges regarding the use of microalgae biomass as a feedstock for biofuels and other biotechnological applications is to find an economical and effective way to separate the microalgae biomass and use it in commercial applications. In order to use the biomass, initially it must be extracted from the liquid culture medium and concentrated so that its processing is effective.

According to the literature, the concentration of biomass can contribute 20 to 30% to the total costs of a biofuel production process, with the total separation time being one of the main factors responsible for the high cost of the conventional techniques, such as centrifugation, filtration and flocculation. In addition, the high recovery cost is associated with the small size of the cells and the low biomass concentrations that do not allow conventional separation methods, such as filtration and centrifugation, to be used, since for such conditions high amounts of energy are required.

The expansion of microalgae production to obtain bioproducts depends on a series of factors that can make the processes involved in obtaining biomass (“upstream”) and its refining (“downstream”) feasible. One of the critical aspects for achieving economicity is the collection step (separation).

In the search for a more economical and effective way to collect the microalgae biomass produced, there is a need to find a low energy and reduced total time process. Sedimentation, even after flocculation, is relatively slow, which leads to long processing times. Furthermore, most polymeric flocculants still have a relatively high price and limited efficiency in removing suspended solids. Therefore, the use of magnetic nanoparticles began to be explored as an alternative or supplement to the use of conventional coagulating and flocculants agents.

The main advantages of magnetic nanoparticles are the high surface area of nanometric materials, which lead to greater removal efficiency, the rapid separation of adsorbed solids through the application of a magnetic field and reuse, facilitated by the magnetic recovery of the flocculant agent. However, the high costs of the chemical processes for the production of synthetic magnetic nanoparticles and the environmental impacts—mainly related to the coating of the nanoparticles—are the main limitations to their use in the removal of suspended solids.

For example, Zhu et al. (2017) describe a method for recovering microalgae using uncoated iron oxide (Fe₃O₄) or yttrium oxide iron (Y₃Fe₅O₁₂) magnetic nanoparticles. Although the nanoparticles from Zhu et al. promote microalgae recovery, the use of such uncoated forms is not sustainable. It is known that synthetic nanoparticles have a shorter useful life due to the absence of a coating (Klektoka et al., 2021). Added to this, due to the natural degradation of these nanoparticles, there is a high release of di- and trivalent iron cations.

The synthetic magnetic nanoparticles are crystallographically imperfect with poorly defined shape and faces, which facilitates their oxidation, loss of magnetic property and dissolution of the nanocrystal. Several authors point out the degradation/dissolution of magnetite nanoparticles in solutions with an acidic pH as a necessary condition for cell separation (Ike and Duke, 2018). Such an abrupt release of iron results in toxic effects to target cells, which can impair downstream applications during processing that require a viable cell or without extravasation of cytoplasmic content (Lei et al., 2016).

Furthermore, the increase in iron in microalgae production tanks tends to cause an increase in the bacterial community, including pathogens, and competition relationships, which would be an additional problem on an industrial scale by reducing productivity (Fuentes et al., 2016; Xiao et al., 2021).

In turn, document KR20140133311 discloses a method for recovering microalgae using magnetic nanoparticles coated with chitosan. Coating with the polysaccharide supposedly allows the preservation of the nanoparticle and its reuse in repeated procedures.

However, coating the nanoparticles with chitosan employs Pluronic F-127—a non-ionic hydrophilic surfactant copolymer. This is a compound with known toxicity to several marine organisms (Hering et al., 2020; Rahdar et al., 2020). The process of coating magnetic nanoparticles with chitosan also makes use of sodium tripolyphosphate (STPP), an aquatic pollutant that is not removed by water treatment plants and will act in the eutrophication of fresh water ecosystems (Lu et al., 2014).

Furthermore, chitosan is a cationic polysaccharide produced from the deacetylation of chitin, a polysaccharide present in the exoskeleton of arthropods and in the cell wall of fungi. Despite being a natural coating, that is, with low environmental impact, its production is costly and, therefore, has a deleterious impact on the economic viability of microalgae collection processes that use this type of nanoparticle.

The limitation of flocculants already described in promoting the collection of marine microalgae in high salinity media (Vandamme et al., 2010; Roselet et al., 2017) assesses the issues of sustainability and economic viability. In these cases, the loss of efficiency of the flocculant or the excessive increase in its concentration is described (Lam et al., 2015), so that there is an effect of flocculation of the microalgae.

With the need to optimize the concentration process of cells for their recovery, aiming at reducing the cost and the sustainability of derivative bioproducts (e.g., biofuels, biofertilizers or biostimulants), the use of magnetic nanoparticles of biological origin (NMOBs) was proposed. The present invention describes the use of magnetic nanoparticles of biological origin (NMOBs), which already have a natural coating, which greatly reduces the cost of production, in addition to being a green process without the generation of toxic waste.

The use of NMOBs promoted the concentration and recovery of target cells in seconds in the bench experiments, which indicates great potential to solve the reported technical problem.

NMOBs are presented, therefore, as an innovative tool, since they already naturally have a biological membrane, which can act in the most efficient interaction between nanoparticle and oil, in addition to having chemical and physical properties that make them highly stable. In addition, they are produced by the growth of bacteria in fermenters, being sustainable and low-cost processes compared to synthetic magnetic nanoparticles with similar properties.

### SUMMARY OF THE INVENTION

The invention employs magnetic nanoparticles of bacterial origin to enhance the recovery of microalgae from a suspension, be it a cell culture, a saltwater suspension, a fresh water suspension or an effluent.

In one embodiment, the invention provides for the use of magnetic nanoparticles of bacterial origin (NMOBs) to recover microalgae present in a suspension.

In a preferred embodiment, the use of NMOBs provided for by the invention is for the recovery of microalgae from a suspension with a salt concentration equal to or greater than 5 g/mL.

According to the invention, microalgae can be selected from Chlorella sp., Crypthecodinium sp., Cylindrotheca sp., Desmodesmus sp., Dunaliella sp., Isochrysis sp., Monoraphidium sp., Nannochloris sp; Nannochloropsis, Neochloris sp. Nitzschia sp., Phaeodactylum sp., Porphyridium sp., Scenedesmus sp., Schizochytrium sp., Tetraselmis sp. or any other genera known to be useful for the production of bio-oils.

In another embodiment, the invention provides a process for recovering microalgae in a suspension using NMOBs as a flocculant agent. The process of the invention comprises protonating the cell surface of the microalgae, adding magnetic nanoparticles of bacterial origin to the suspension and applying an external magnetic charge, thus promoting flocculation of the microalgae.

In one aspect, the process of the invention provides that the microalgae suspension is selected from: a microalgae culture, a source of salt water, a source of fresh water or an effluent.

In another aspect, the process of the invention provides that the suspension has a salt concentration equal to or greater than 5 g/mL.

Further, in an additional aspect, said process provides that the cell surface of the microalgae is protonated by adding an acidic buffer to the suspension.

In another aspect, the process comprises the addition of NMOBs to the microalgae suspension at a concentration between 50 and 100 μg/mL.

Finally, according to the process of the invention, microalgae can be selected from Chlorella sp., Crypthecodinium sp., Cylindrotheca sp., Desmodesmus sp., Dunaliella sp., Isochrysis sp., Monoraphidium sp., Nannochloris sp., Nannochloropsis Neochloris sp., Nitzschia sp., Phaeodactylum sp., Porphyridium sp., Scenedesmus sp., Schizochytrium sp., Tetraselmis sp. or any other genera known to be useful for the production of bio-oils.

### BRIEF DESCRIPTION OF THE FIGURES

FIG. 1 illustrates octahedral NMOBs produced by Mf. australis strain IT-1 (A) and prismatic NMOBs produced by Mv. blakemorei strain MV-1^{T} (B).

FIG. 2 results from the evaluation of the magnetic concentration of Scenedesmus sp. by using octahedral NMOBs and applying an external magnetic field. (A) Cells in the supernatant after magnetic recovery. (B) Cell recovery rate as a function of pH.

FIG. 3 results from the evaluation of the magnetic concentration of Desmodesmus sp. by using octahedral NMOBs and applying an external magnetic field. (A) Cells in the supernatant after magnetic recovery. (B) Cell recovery rate as a function of pH.

FIG. 4 results from the evaluation of the magnetic concentration of Monoraphidium sp. by using octahedral NMOBs and applying an external magnetic field. (A) Cells in the supernatant after magnetic recovery. (B) Cell recovery rate as a function of pH.

FIG. 5 results from the evaluation of the magnetic concentration of Nannochloropsis sp. by using octahedral NMOBs and applying an external magnetic field. (A) Cells in the supernatant after magnetic recovery. (B) Cell recovery rate as a function of pH.

FIG. 6 results from the evaluation of the magnetic concentration of Dunaliella sp. by using octahedral NMOBs and applying an external magnetic field. (A) Cells in the supernatant after magnetic recovery. (B) Cell recovery rate as a function of pH.

FIG. 7 presents images obtained by videos of culture of Desmodesmus sp. containing or not octahedral NMOBs before applying the magnetic field (A) and after applying the magnetic field for 20 seconds (B). Note the clarification of the supernatant from the tube containing the cell culture and NMOBs in (B) after applying the magnetic field compared to the control (culture without the addition of NMOBs). In (B), it is possible to observe the accumulation of cells on the tube wall next to the magnet in the tube containing the culture and NMOBs. The same result was observed for the other evaluated cultures, except for Monoraphidium sp.

FIG. 8 presents photomicrographs derived from differential interferential contrast microscopy of cultures of Desmodesmus sp. submitted to different pH conditions and to octahedral NMOBs (A-D). Control conditions (no addition of NMOBs) are represented (E-H).

FIG. 9 presents photomicrographs derived from differential interferential contrast microscopy of cultures of Scenedesmus sp. submitted to different pH conditions and to octahedral NMOBs (A-D). Control conditions (no addition of NMOBs) are plotted (E-H).

FIG. 10 presents photomicrographs derived from differential interferential contrast microscopy of cultures of Monoraphidium sp. submitted to different pH conditions and to octahedral NMOBs (A-D). Control conditions (no addition of NMOBs) are represented (E-H).

FIG. 11 results from the evaluation of the magnetic concentration of Scenedesmus sp. by using prismatic NMOBs and applying an external magnetic field. (A) Cells in the supernatant after magnetic recovery. (B) Cell recovery rate as a function of pH.

FIG. 12 results from the evaluation of the magnetic concentration of Monoraphidium sp. by using prismatic NMOBs and applying an external magnetic field. (A) Cells in the supernatant after magnetic recovery. (B) Cell recovery rate as a function of pH.

FIG. 13 results from the evaluation of the magnetic concentration of Scenedesmus sp. by using NPSs and applying an external magnetic field. (A) Cells in the supernatant after magnetic recovery. (B) Cell recovery rate as a function of pH.

FIG. 14 results from the evaluation of the magnetic concentration of Desmodesmus sp. by using NPSs and applying an external magnetic field. (A) Cells in the supernatant after magnetic recovery. (B) Cell recovery rate as a function of pH.

FIG. 15 results from the evaluation of the magnetic concentration of Nannochloropsis sp. by using NPSs and applying an external magnetic field. (A) Cells in the supernatant after magnetic recovery. (B) Cell recovery rate as a function of pH.

FIG. 16 results from the evaluation of the magnetic concentration of Monoraphidium sp. by using NPSs and applying an external magnetic field. (A) Cells in the supernatant after magnetic recovery. (B) Cell recovery rate as a function of pH.

FIG. 17 results from the evaluation of the magnetic concentration of Dunaliella sp. by using NPSs and applying an external magnetic field. (A) Cells in the supernatant after magnetic recovery. (B) Cell recovery rate as a function of pH.

FIG. 18 illustrates the behavior of a culture of Scenedesmus sp., containing or not NPSs, before applying the magnetic field (A) and after applying the magnetic field for 20 seconds (B). It should be noted that there is no clarification of the supernatant from the tube containing the cell culture and NPSs in (B) after applying the magnetic field compared to the control. The same result was observed for the other evaluated cultures.

FIG. 19 illustrates the behavior of magnetite NPSs. (A) Synthetic nanoparticles obtained by coprecipitation and used in the magnetic collection essays for comparison with the efficiency of NMOBs. (B) Synthetic magnetite nanoparticles similar to those used in reference D1 (Leon-Reyes et al., 2014).

FIG. 20 demonstrates the recovery rate of microalgae of the genus Nannochloropsis. A. Statistically significant recovery rate of 23.01+4.94% and (***) 17.31+3.27% (**) in citrate buffer with pH 3.1 and 6.4, respectively. B. Statistically significant recovery rate in pH 3.1 citrate buffer only (**). C. There was no statistically significant recovery when using the flocculant (4.30+1.81%) in the culture condition with pH 8.0.

FIG. 21A shows the response surface of the recovery rate of Nannochloropsis sp. using NMOBs varying the conditions of pH and NaCl concentration. There are shown the optimal point (orange arrowhead), the acidification point of the culture condition with 40 g NaCl/L (red arrowhead) and the acidification point in the central condition of NaCl concentration (20 g/L; blue arrow head).

FIG. 21B shows the response surface of the recovery rate of Nannochloropsis sp. using NMOBs varying the conditions of pH and NaCl concentration.

FIG. 21C shows the response surface of the recovery rate of Nannochloropsis sp. using NMOBs varying the conditions of pH and NaCl concentration.

FIG. 21D shows the two-dimensional projection of the response surface of the recovery rate of Nannochloropsis sp. using NMOBs varying the conditions of pH and NaCl concentration.

FIG. 22 illustrates the collection and magnetic separation of microalgae of the genus Nannochloropsis. Essays carried out with cells resuspended in citrate buffer with pH fixed at 3.1 and variations in the salinity condition. The magnetic field was generated by positioning a neodymium-boron magnet on the side of the tube. A, B. and C. Essays on Nannochloropsis sp. after the magnetic concentration of the biomass on the tube wall next to the magnet. It was possible to observe a response to the magnetic field generated under the conditions of 0, 20 and 40 g/L. In each image, the control tube with the microalgae species without the addition of NMOBs is positioned on the left, and the treatment condition with the addition of NMOBs is positioned on the right.

### DETAILED DESCRIPTION OF THE INVENTION

Unless otherwise specified, terms used throughout this specification have their common meanings in the art, within the context of the disclosure and the specific context in which each term is used. Certain terms that are used to describe the disclosure are discussed below, or elsewhere in the specification, to provide additional guidance to the practitioner in relation to describing the disclosure. Publications cited herein and the material to which they are cited are specifically incorporated by reference in their entirety.

It will be appreciated that the same thing can be said in different ways. Consequently, alternative language and synonyms may be used for any one or more of the terms discussed herein. No special meaning should be placed on whether a term is elaborated or discussed here. Synonyms for certain terms are provided, but the exemplification of some synonyms does not exclude the potential use of others that may not be listed here.

As used in this description, the term nanoparticle denotes structures or particles of matter having size in the nanometric (nm) scale. Therefore, in the context of the invention, a nanoparticle generally has an average particle size of less than 1000 nm, for example about 1 nm, about 10 nm, about 100 nm, about 250 nm, about 500 nm, or from 500 to 999 nm.

The expression “magnetic nanoparticle”, in the context of this invention, denotes a nanoparticle having magnetic properties. “Magnetic nanoparticles” can be composed of metals, metal alloys, sulfides and/or metal oxides of one or more metallic elements, such as, for example, iron, nickel, cobalt, manganese, bismuth, zinc, gadolinium, strontium, copper, silver, gold, zinc, cadmium, silver, aluminum or others recognized by the person skilled in the art.

A magnetic nanoparticle of biological origin, also referred to as NMOB description, concerns the structure formed by the isolation of magnetosomes, an organelle present in magnetotactic bacteria. Such magnetosomes are chains of granules of metallic oxides or sulfides, usually of uniform size, profiled in the cytoplasm of magnetotactic bacteria. Most of the time, they are composed of iron oxide (Fe₃O₄), known as magnetite, or iron sulfide (Fe₃S₄), known as greigite). It is the magnetosomes that provide magnetotactic bacteria with the ability to move in response to magnetic fields.

Several types of bacteria, including cocci, bacilli, vibrions and spirilla have the ability to perform biomineralization—a process by which minerals that make up magnetosomes are formed. In nature, magnetosomes can range in size from 35 to 250 nm and can assume different shapes, such as, for example, cube-octahedral, prismatic or parallelepiped.

One of the main advantages of magnetosomes for microalgae recovery processes is the phospholipid membrane that naturally surrounds them, providing to natural magnetosomes compatibility with biological systems. In general, the casing of magnetosomes is composed of 98% lipids and 2% other components, including proteins. Most lipids, particularly phospholipids (Yoshino et al., 2008).

Magnetosomes can be obtained from wild-type cells as well as from genetically modified cells to artificially produce the same and/or to synthesize magnetosomes with artificially introduced functionalities (e.g., vectoring molecules such as, for example, antibodies). For example, document U.S. Pat. No. 6,251,365B1 teaches the isolation of magnetosomes from the bacterium Magnetospirillum gryphiswaldense. Yoshino et al. (2008) in turn teach that a Magnetospirillum magneticum bacterium of the strain AMB-1 can be genetically manipulated to express chimeric proteins containing a protein A domain in the magnetosome membrane, improving their use for the recovery of cells opsonized with antibodies. Another possibility, as taught by US20100297022A1, is to artificially promote the expression of magnetosomes in eukaryotic cells and extract magnetic nanoparticles therefrom.

In the context of this invention, the term “microalgae” represents a group of unicellular, autotrophic, prokaryotic or eukaryotic microorganisms. Several genera of microorganisms fall under the classification of microalgae, such as Chlorella sp., Crypthecodinium sp., Cylindrotheca sp., Desmodesmus sp., Dunaliella sp., Isochrysis sp., Monoraphidium sp., Nannochloris sp., Nannochloropsis, Neochloris sp., Nitzschia sp., Phaeodactylum sp; Porphyridium sp., Scenedesmus sp., Schizochytrium sp., Tetraselmis sp., Botryococcus sp., Spirulina sp., Monodus sp., Chlamydomonas sp. or any other genera known to be useful for the production of bio-oils.

Microalgae can be cultured or obtained from a natural source. For example, microalgae can be cultivated by techniques known by a technician skilled on the subject, such as those illustratively disclosed in Yoshino et al. (2008), U.S. Pat. No. 6,251,365B1 and US20100297022A1, incorporated herein by reference. Further, microalgae can be isolated from suspensions obtained in nature, such as sea water, fresh water sources and/or organic effluents.

The invention is particularly applicable to the isolation of microalgae from suspensions with high salinity. In the context of the present invention, the expression “high salinity” and the analogous term “hypersaline” refer to aqueous solutions of any nature with a salt concentration (e.g., NaCl) equal to or greater than at least 5 g/L, at least 10 g/L, at least 15 g/L, at least 20 g/L, at least 30 g/L or at least 40 g/L.

By the present invention, microalgae can be isolated from a suspension in a simple manner. With the use of NMOBs, it is possible to isolate microalgae from a culture with about 80% efficiency in a small number of steps, namely: (1) charge conflict resolution, (2) incubation with magnetic nanoparticles of biological origin, and (3) application of external magnetic field.

Since the lipid membrane that coats the NMOBs tends to have a net negative charge, the microalgae surface can be protonated by acidifying the medium with an acidic character buffer, such as citrate buffer, to favor electrostatic interaction between microalgae and NMOBs. The pH of the buffer can be set to at least 3.0, at least 4.0, at least 5.0 or at least 6.0.

Then, the culture microalgae are incubated with the NMOBs for a time that can vary between about 1 and about 20 minutes. In this step, NMOBs are added to the microalgae culture at a concentration that can vary according to the number of microalgae cells in the culture. For example, for microalgae cultures with cell density from 10³ cells/mL, NMOBs can be added to the culture in sufficient quantity to reach a final concentration of at least 10 μg×mL-1, at least 50 μg×mL-1, at least 80 μg×mL-1 or at least 100 μg×mL-1.

Finally, an external magnetic force source is applied to the culture of microalgae incubated with NMOBS, promoting flocculation of the microalgae and thus favoring their separation from the medium. The application of an alternating magnetic field in a later step can damage the cell wall of microalgae and facilitate the extraction of lipids therefrom. The magnetic recovery process can have its efficiency increased by the association with electroflocculation.

In the context of the present invention, the term “flocculation” and variants refer to the process by which small particles present in a suspension aggregate, forming flocs. Such flocs can then be recovered by, for example, decanting. The flocculation phenomenon can be induced by a chemical (e.g., chemical flocculant) or physical (e.g., electroflocculation) agent. The present invention provides a physical means of promoting microalgae flocculation through the use of NMOBs, which are responsive to the application of an external magnetic force.

Subsequently, the NMOBs can be recovered and separated from the microalgae by a chemical or physical process, ensuring high concentration and purity of the material of interest, and the possibility of reusing the NMOBs, which reduces costs and is a characteristic of sustainable processes.

Next, the invention will be illustrated by means of examples of embodiments, which do not exhaust all the possibilities achievable by the inventive concept described herein, but represent all the embodiments. The examples below are presented in order to provide to the technician skilled on the subject a complete description of how the compositions and methods of this invention are prepared, evaluated and used. A technician skilled on the subject, in light of the present disclosure, will recognize that many changes can be made to the specific embodiments that are disclosed and still obtain a similar or equivalent result, without departing from the spirit and scope of the invention.

### EXAMPLES

### Example 1—Obtention of Bacterial Magnetic Nanoparticles Culture of Magnetotactic Bacteria

In this step, magnetotactic bacteria Magnetovibrio blakemorei, strain MV-1^{T}, and Magnetofaba australis, strain IT-1, were used. Volumes corresponding to a final concentration of 10⁸ cells×mL-1 were inoculated into a 5 L benchtop bioreactor (2 L working volume) (Minifors, Infors HT—Basel, Switzerland) containing fresh growth medium. Culture parameters were established as follows: pH 7.0 (adjusted with 1.0 N NaOH or HCl), stirring speed 100 RPM, 28° C. Prior to inoculum, the anaerobic condition was achieved by purging nitrogen (N₂) in sterile, fresh media until the O₂ sensor reading reached zero. Then, the medium was purged with N₂O for 15 min in the case of Magnetovibrio blakemorei strain MV-1^{T} and O₂ was allowed to enter, being maintained at 0.5% for Magnetofaba australis strain IT-1. Cultures were carried out for 120 hours.

### Extraction and Purification of Bacterial Magnetic Nanoparticles (Magnetosomes)

At the end of the period of growth in the bioreactor, the cells were collected by centrifugation at 6100×g at 4 ºC for 15 min. Cell pellets were washed and resuspended in 15 ml of Hepes buffer (20 mM, pH 6.8). Then, the cells were subjected to lysis by ultrasonic disruption in a sonicator with tips (VCX500, Sonics, Newtown, USA) at an amplitude of 40%, frequency 20 kHz, in 60 cycles of 30 s with intervals of 30 s. The NMOBs were magnetically concentrated by a boron-neodymium magnet attached to the outside of the tube for 12 h at 4 ºC. The crystals were transferred to 1.5 mL polypropylene tubes and resuspended in Hepes buffer (10 mM, pH 6.8) with NaCl (200 mM). Next, the crystals were washed in an ultrasound bath (Branson 2200, Emerson, Rochester, USA) for 4 cycles of 30 min, with magnetic concentration and buffer exchange at each cycle. Note: the cells can be recovered by using the magnetic field, but for the ease in the laboratory, centrifugation was performed.

### Characterization of Nanoparticles

Cells and purified NMOBs were added onto copper grids coated with polyvinyl formal and carbon support film, being air-dried for observation in a transmission electron microscope (FEI Morgagni, Hillsboro, USA) operating at 80 kV in a direct increase of 36,000 and 89,000 times for each case, respectively. The average number of NMOBs per cell is determined by the average number of NMOBs in 30 cells for each sampling point. The mass of extracted NMOBs was quantified by the method described by Yoshino et al. (2008).

### Example 2—Magnetic Recovery of Microalgae

For the microalgae magnetic collection essays, the following cells were used: Scenedesmus sp., Monoraphidium sp., Desmodesmus sp., Monoraphidium sp., Nannochloropsis sp. and Dunaliella sp. Cells were cultivated in the modified BG11 culture medium. As a flocculation strategy to solve the conflict between surface charges, the microalgae surface was protonated using: (i) citrate buffer prepared at pH ranges 3.1, 4.8 and 6.4; and (ii) phosphate buffer prepared at pH 7.2. Samples were prepared in triplicate for each condition and cell type. Samples were treated with prismatic and octahedral NMOBs at a final concentration of 80 μg×mL-1 and incubated for 5 min. The samples were read in a spectrophotometer (Biospectro SP-22, Paraná, Brazil) for optical density at 680 nm. Parallel to this, the samples were also observed in optical microscopy in differential interferential contrast. The same experiment was carried out with synthetic magnetic nanoparticles produced by the coprecipitation method described by Santos et al. (2018).

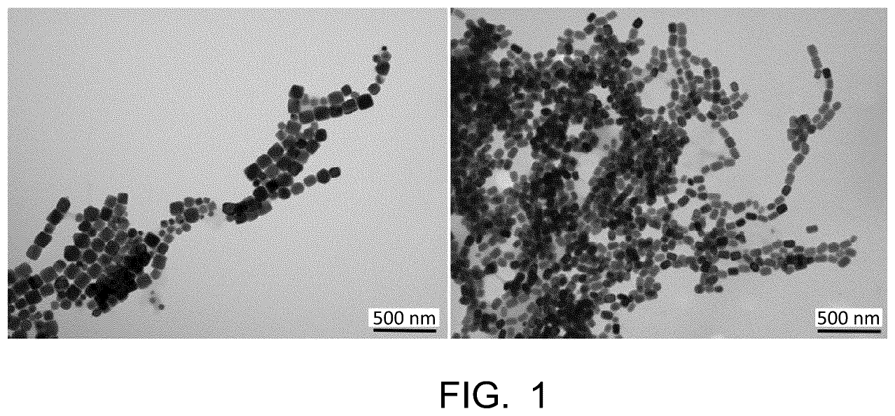

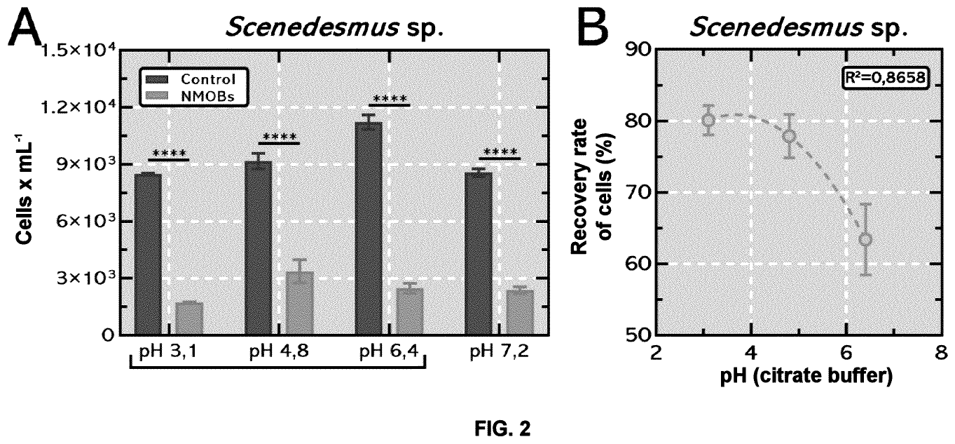

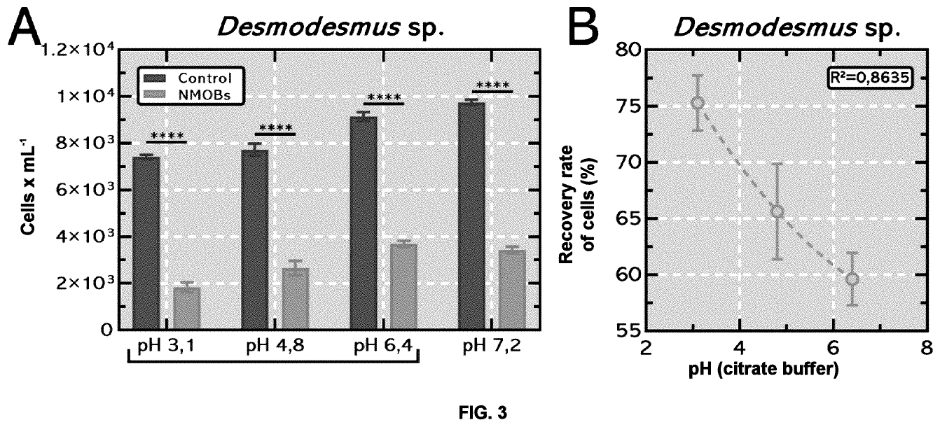

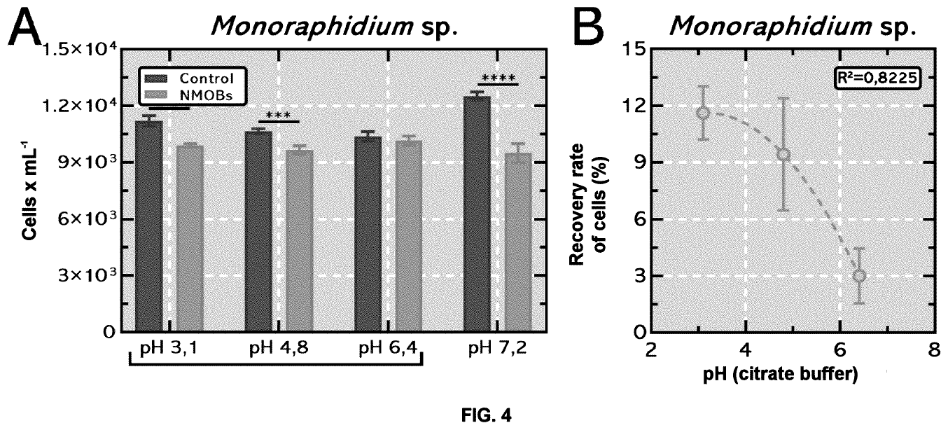

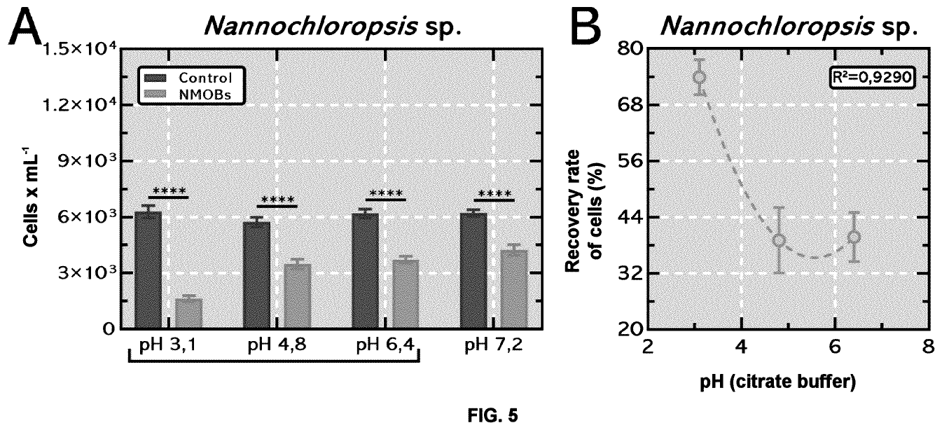

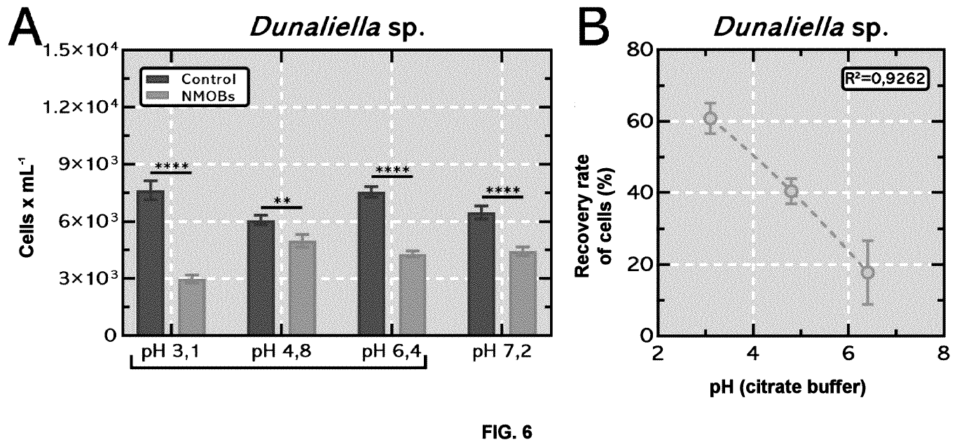

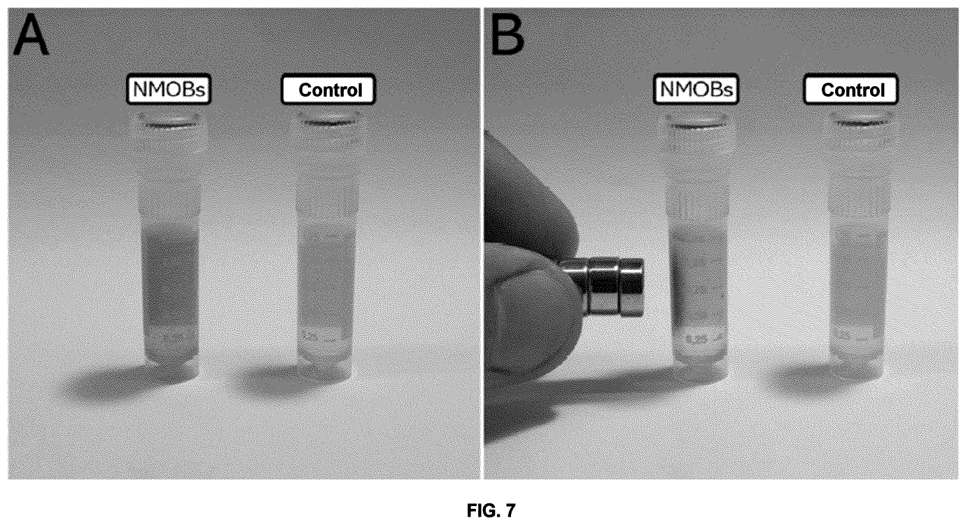

In the microalgae magnetic collection essays, the use of octahedral NMOBs (FIG. 1A) promoted the removal of cells in the supernatant in cultures of Scenedesmus sp. (FIG. 2), Desmodesmus sp. (FIG. 3), Monoraphidium sp. (FIG. 4), Nannochloropsis sp. (FIG. 5) and Dunaliella sp. (FIG. 6). In all cases, the efficiency of the cell recovery rate was directly related to the acidification of the medium, reaching values close to 70% of cell recovery within 5 minutes of application of an external magnetic field. In fact, the application of a magnet for 20 seconds and video recording (FIG. 7) indicates that the recovery time is much lower than the 5 minutes established in the methodology. Exceptions to this recovery rate were found for Dunaliella sp. (best recovery rate close to 60%) and Monoraphidium sp. (best recovery rate close to 12%) species.

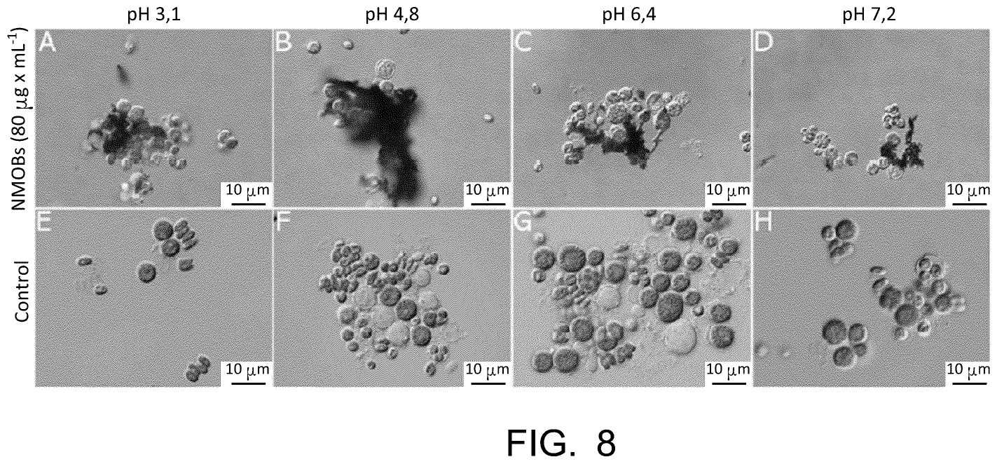

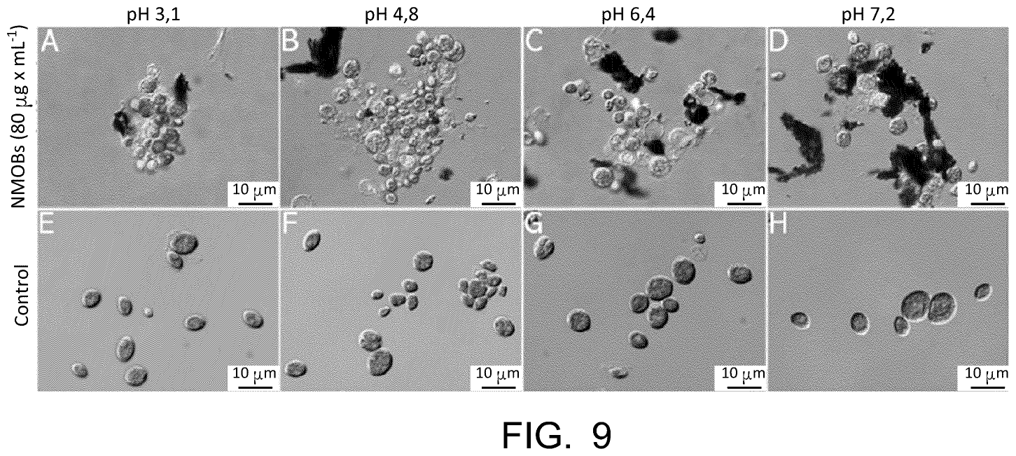

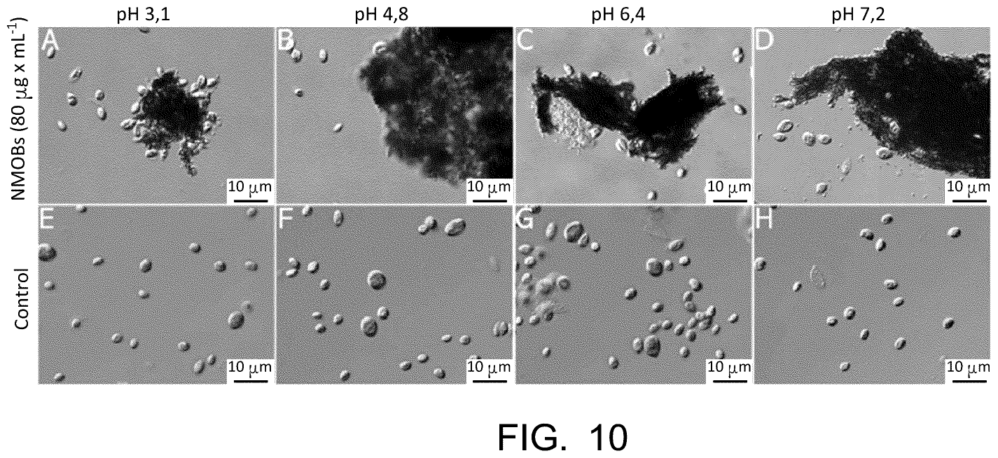

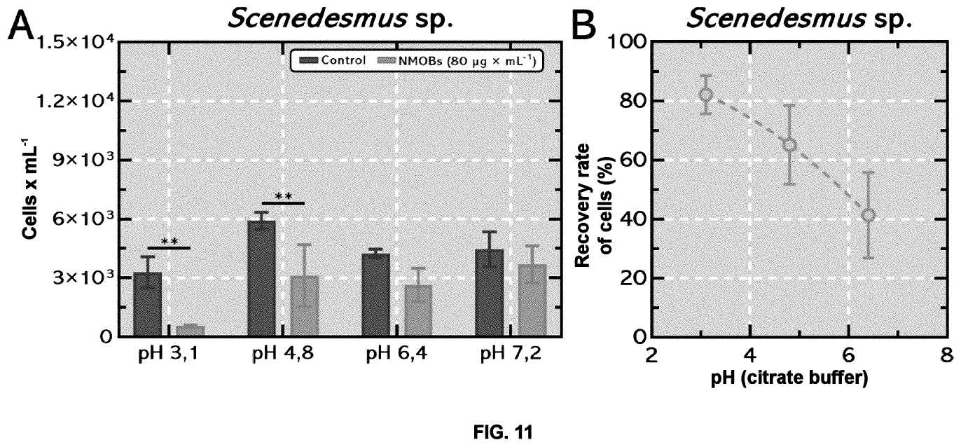

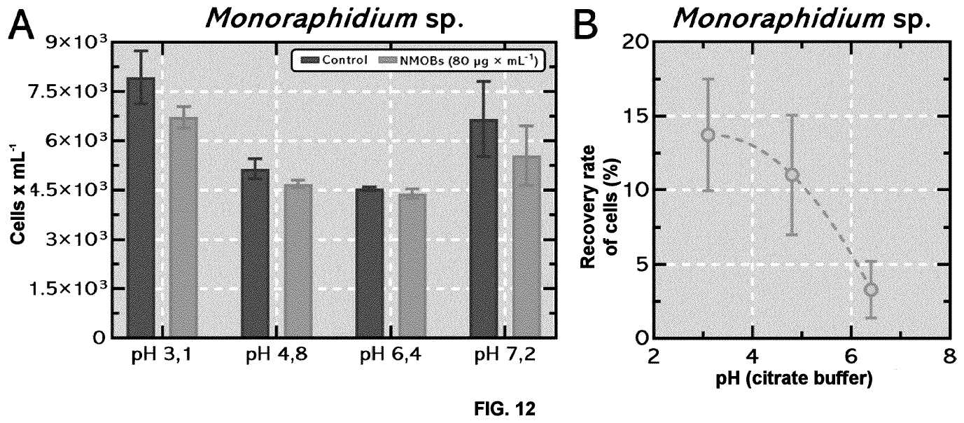

The observation of the interaction of octahedral NMOBs with cells by optical microscopy showed the addition of magnetic nanoparticles promotes aggregation of cells with the same (FIGS. 8 and 9). Except for Monoraphidium sp., which seems to exclude NMOBs, clustering them in isolation (FIG. 10), which shows that there is some physicochemical characteristic of this cell that prevents interaction with NMOBs. The exact reason for this phenomenon is not described in the literature. However, a detailed study and experimentation may solve this issue and enable the use of NMOBs in the collection of Monoraphidium sp. In the microalgae collection essays using prismatic NMOBs (FIG. 1B), Scenedesmus sp. and Monoraphidium sp. were used, since these cells showed the best and worst results in the previous evaluation using octahedral NMOBs. Prismatic NMOBs promoted cell collection in cultures of Scenedesmus sp. (FIG. 11) and Monoraphidium sp. (FIG. 12). The efficiency of cell recovery through the application of a magnetic field was higher in the case of the culture of Scenedesmus sp. (82.03+6.55% recovery at pH 3.1), as observed previously using octahedral NMOBs. The efficiency of the cell recovery rate was directly related to the acidification of the medium, reaching values close to 80% of cell recovery in 5 minutes of application of an external magnetic field. Again, it was observed that magnet application for 20 seconds is sufficient to visually determine cell aggregation.

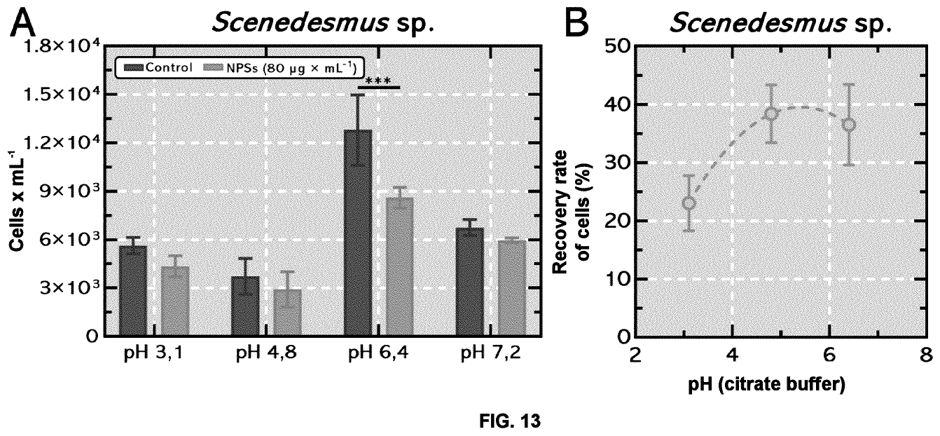

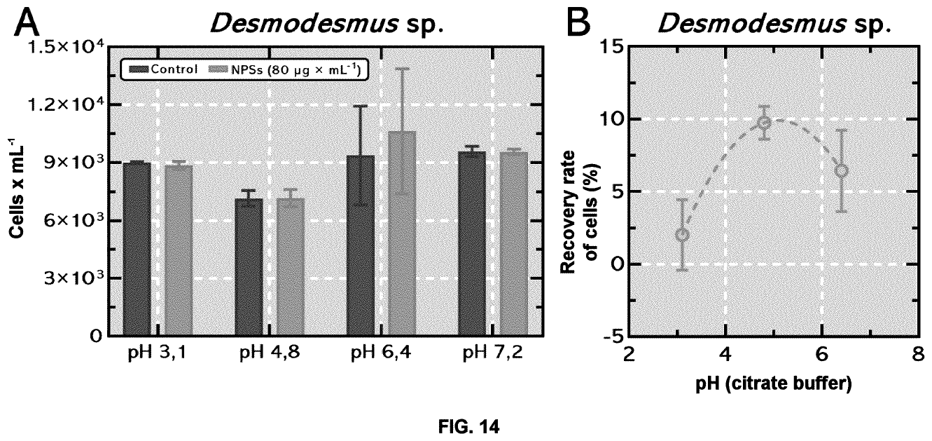

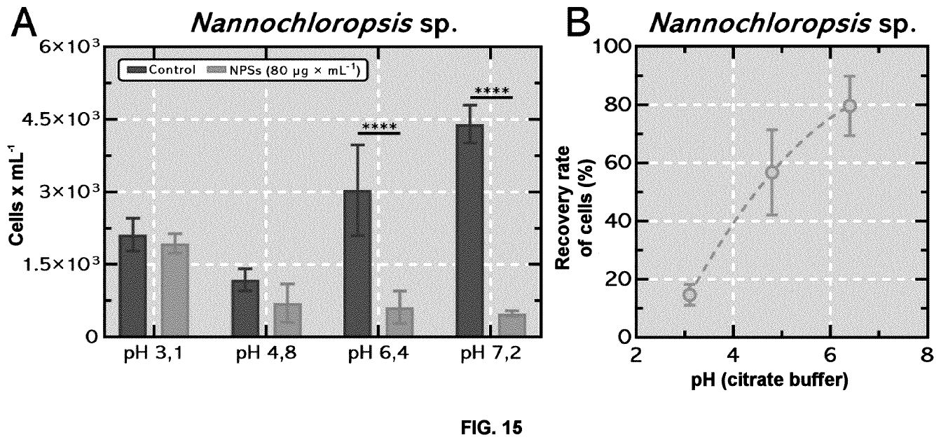

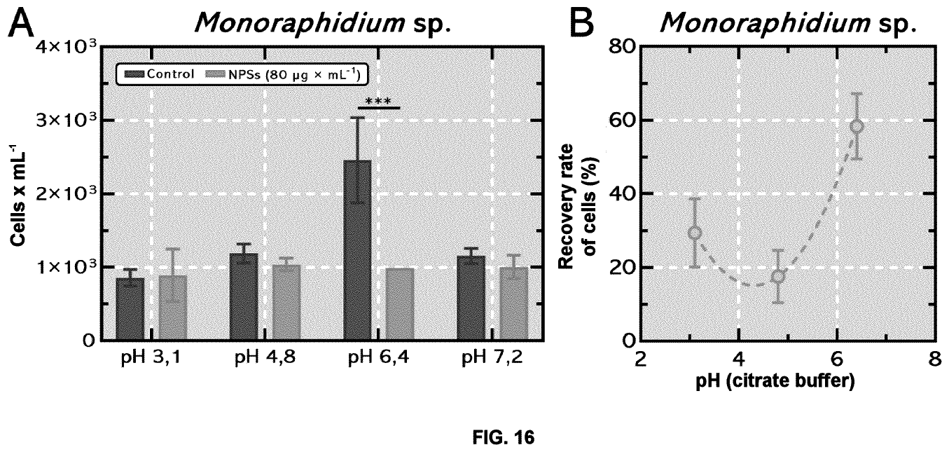

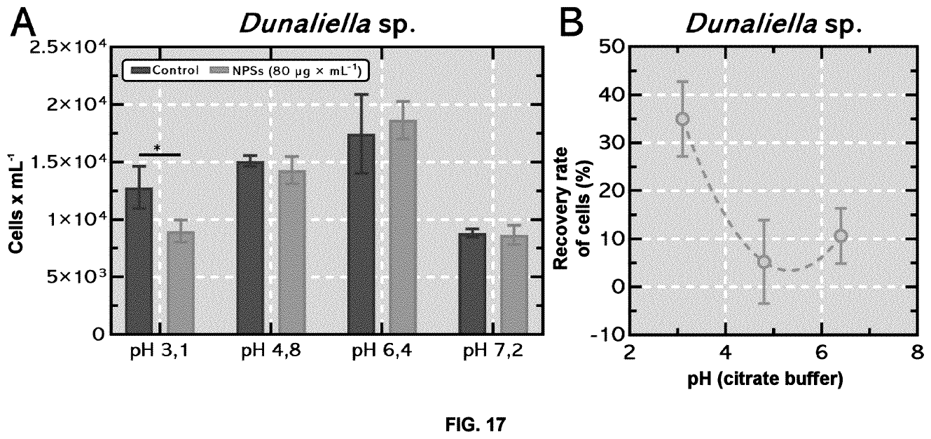

When synthetic magnetic nanoparticles were used, recovery rates lower than octahedral and prismatic NMOBs were observed for Scenedesmus sp. (FIG. 13), Desmodesmus sp. (FIG. 14), Monoraphidium sp. (FIG. 15), Nannochloropsis sp. (FIG. 16) and Dunaliella sp. (FIG. 17).

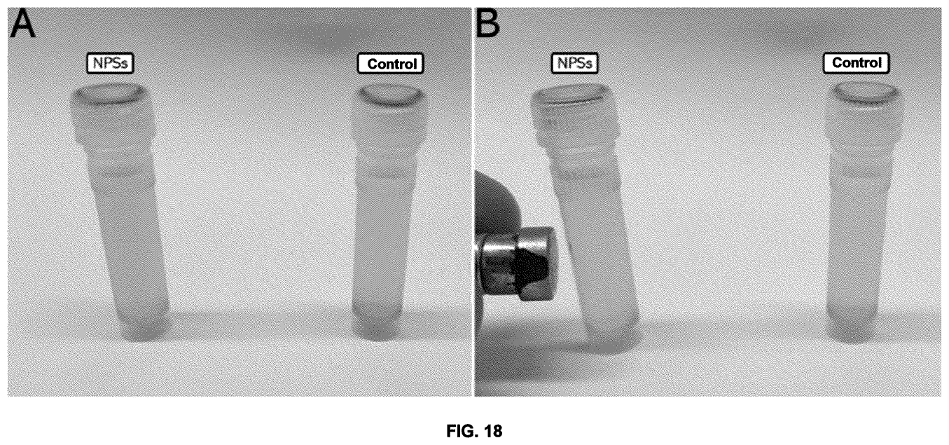

Contradicting the results of microalgae collection based on absorbance as shown above, the visual evaluation of microalgae recovery by synthetic nanoparticles through the application of an external magnetic field showed that there was no agglomeration of cells, which makes magnetic collection unfeasible (example with magnetic collection essay of Scenedesmus sp.; FIG. 18). Although FIG. 18 is a representation of the magnetic collection experiment with the culture of Scenedesmus sp. subjected to magnetic recovery by synthetic nanoparticles, the same result, that is, the absence of agglomeration/concentration of cells when a magnetic field was applied, was repeated in the experiments with the other cell cultures.

It is likely that due to the fact that synthetic nanoparticles are not crystals with well-defined faces and crystallographically perfect, these synthetic magnetic nanoparticles are being dissolved in the medium, which would cause the release of large amounts of iron and cellular changes, including cell lysis. Several authors point out the dissolution of synthetic magnetic nanoparticles of magnetite in solutions with an acidic pH, a necessary condition for cell separation (Ike and Duke, 2018), which causes cell damage, including leakage of cytoplasmic content (Lei et al., 2016).

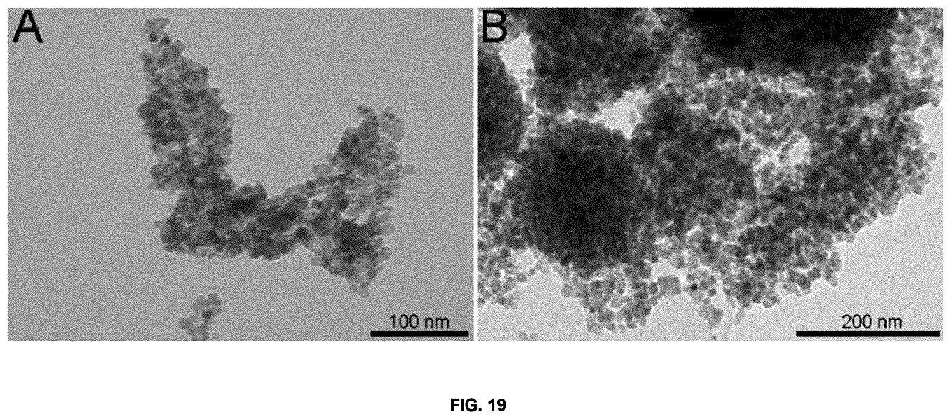

In the case of the experiments shown above, in which the magnetic collection of microalgae is based on absorbance, there is an indication that the dissolution of synthetic magnetic nanoparticles causes “false cell recovery”. It is important to point out that the synthetic nanoparticles used in this experiment are similar to those used in KR20140133311 (FIG. 19). Thus, the performance of NMOBs was significantly superior to that exhibited by synthetic magnetic nanoparticles, as NMOBs are stable and, in fact, promote cell agglomeration/concentration when an external magnetic field (magnet) is applied.

### Example 3—Magnetic Recovery of Microalgae in Hypersaline Media

To demonstrate the ability of NMOBs to promote the recovery of microalgae from media with high salinity, we evaluated the efficiency of magnetic collection of Nannochloropsis sp. cells suspended in a medium with salinity of 40 g/L, comparing the use of nanoparticles and a standard chemical flocculant. Several essays were carried out to investigate the effect of different salinity and pH conditions on the efficiency of NMOBs.

### Establishment of a Mathematical Model for Determining Salinity and pH Conditions

Nannochloropsis sp. cells were cultivated in BG11 medium containing 40 g/L of NaCl. For the preliminary comparative essay of microalgae collection by NMOBS, synthetic magnetic nanoparticles and flocculant (compound kept confidential), the culture was used directly in the essay, adding the same amount of NMOBs and nanoparticles so that the final concentration of 80 μg/mL was reached. The concentration of the flocculant used was 1:5 of the sample. The experiment was carried out at pH 3.1 and 6.4. After the magnetic concentration, in the case of using NMOBs or synthetic magnetic nanoparticles, and waiting for flocculation, in the case of using the flocculant, the supernatant was transferred to a new tube. These samples were read in a spectrophotometer (Biospectro SP-22, Paraná, Brazil) at the optical density at 680 nm to determine the microalgae recovery rate. The synthetic magnetic nanoparticles used were produced by the coprecipitation method described by Santos et al. (2018). Statistical inference by the two-way ANOVA test with Sidak's multiple comparisons post-test was performed using GraphPad Prism software version 8.0 (GraphPad, USA).

For the experimental planning in which different conditions of salinity and pH were tested, the algae obtained in the culture in BG11 medium, as described above, were collected and centrifuged at 3,000 rpm at 24° C. for 10 minutes in polypropylene microtubes of 1.5 mL. Cells were resuspended in citrate buffer solutions at different salinities, as outlined in the experimental planning. The experimental planning carried out was of the Box-Benkhen type for two variables, i.e., pH and salinity (indirectly by the concentration of NaCl present in the medium), in 3 levels for each variable.

The pH ranges were the values of 3.1, 4.8 and 6.4, relative to levels −1, 0 and +1. The salinity ranges, indirectly conferred by the addition of NaCl in the medium, were 0, 20 and 40 g/L, relative to levels −1, 0 and +1, respectively, for Nannochloropsis sp. The planning design, generation of the model matrix, calculation of coefficients and plotting of results were performed using Microsoft Excel 2016 software (Microsoft, USA). The planning followed the model described below:

y=b₀+b₁x₁+b₂x₂+b₁₂x₁x₂+b₁₁x₁²+b₂₂x₂²

After the resuspension of the cells in each condition provided for in the Box-Benkhen type planning, a suspension of prismatic NMOBs extracted from Mv. blakemorei strain MV-1^{T} at a final concentration of 80 μg/mL. The cell recovery efficiency rate was calculated from the difference between the supernatant of the magnetically concentrated material and the control condition without magnetic separation. The difference between each reading considering the concentration step was 5 min. The optical density (OD) of the suspensions was read in a spectrophotometer (Biospectro SP-22, Brazil) at a wavelength of 680 nm. The conversion of 680 nm OD into cell density, i.e., cells/mL, was obtained after previous correlation established by the research group.

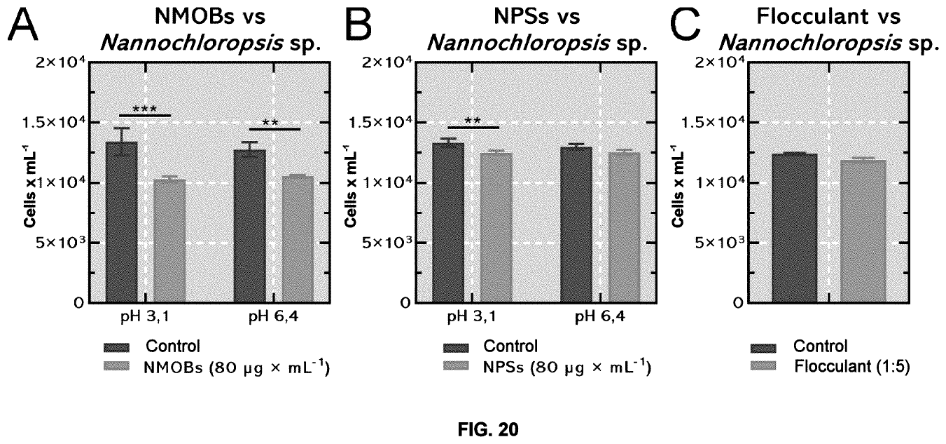

A magnetic collection and separation essay was performed in which the NMOBs were challenged in face of synthetic magnetite nanoparticles and the flocculant, kept confidential, used in the biomass recovery step (FIG. 20). In the hypersaline culture condition of 40 g/L of NaCl and pH 3.1 or 6.4, the NMOBs showed a statistically significant recovery rate of 23.01+4.94% and (***) 17.31+3.27% (**), respectively (FIG. 20A). Synthetic nanoparticles obtained a statistically significant recovery rate of 6.22+2.88% (**) at pH 3.1 (FIG. 20B). There was no statistically significant recovery when using the flocculant (4.30+1.81%) in the culture condition at pH 8.0 (FIG. 20C).

Recovery of Nannochloropsis sp. Under Varied Conditions of Salinity and pH

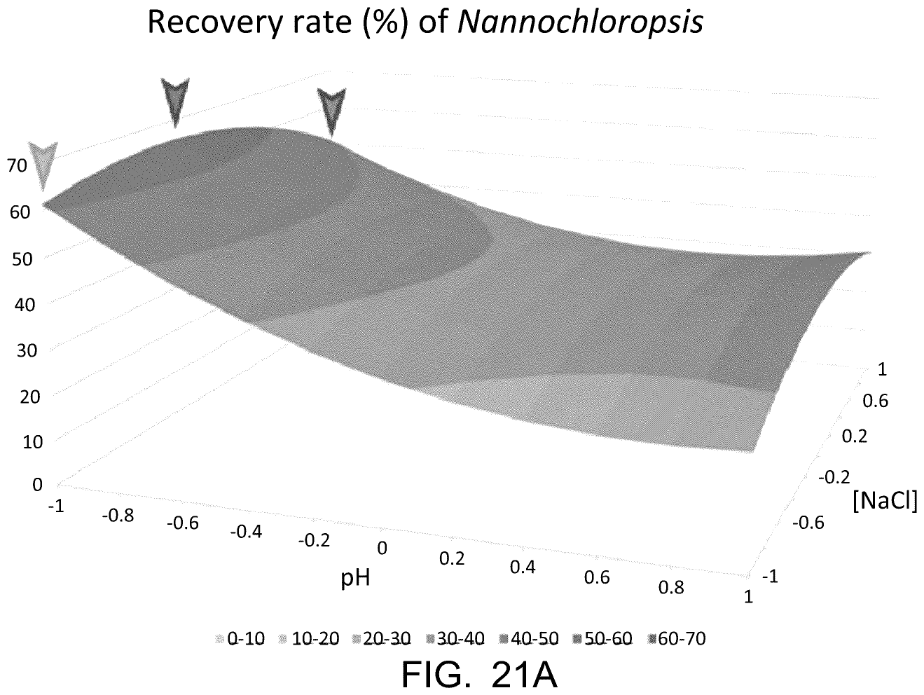

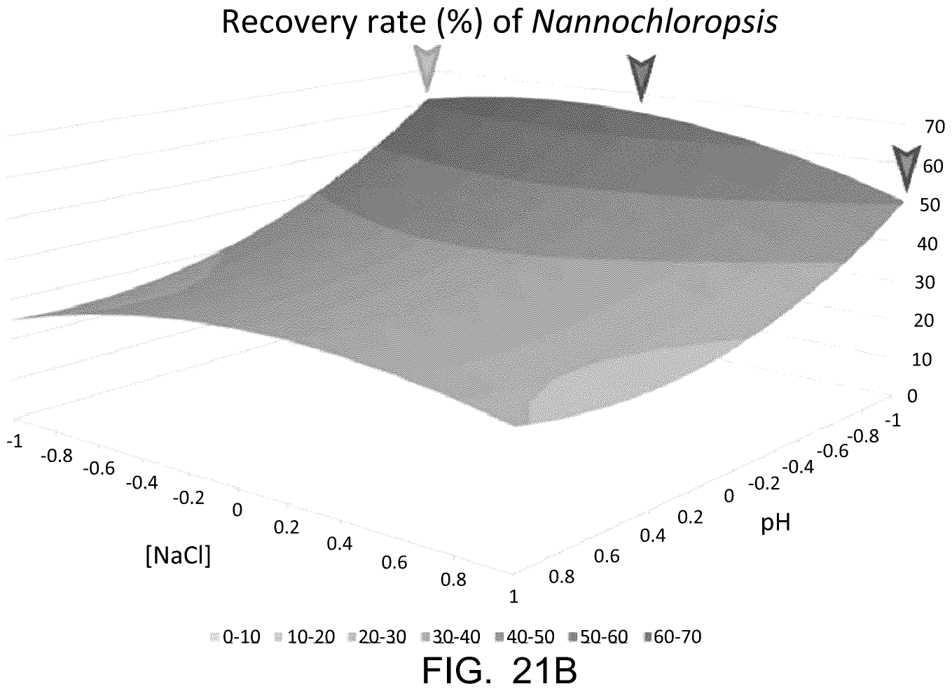

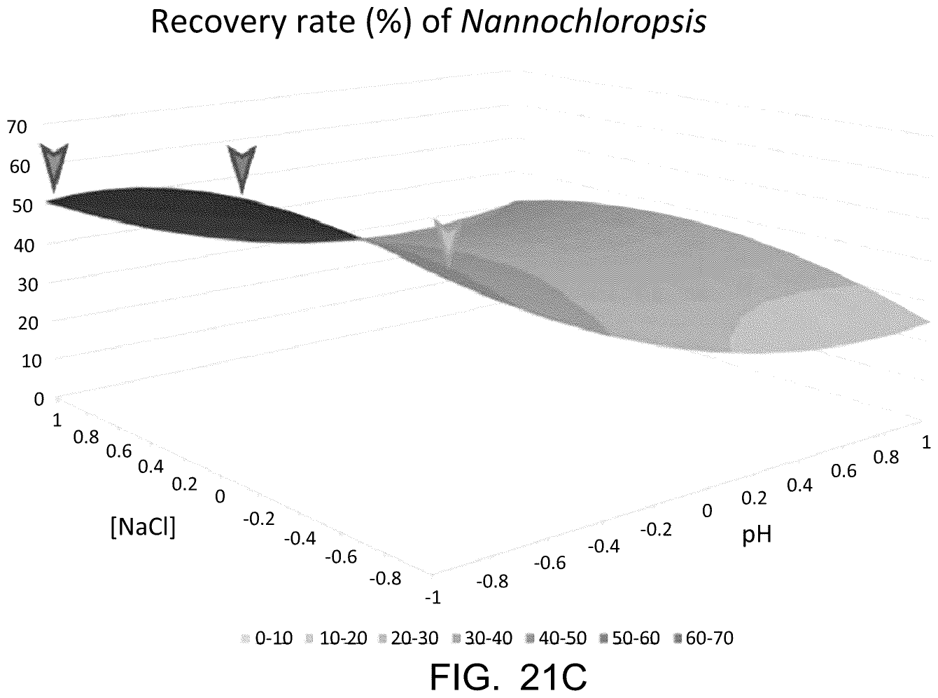

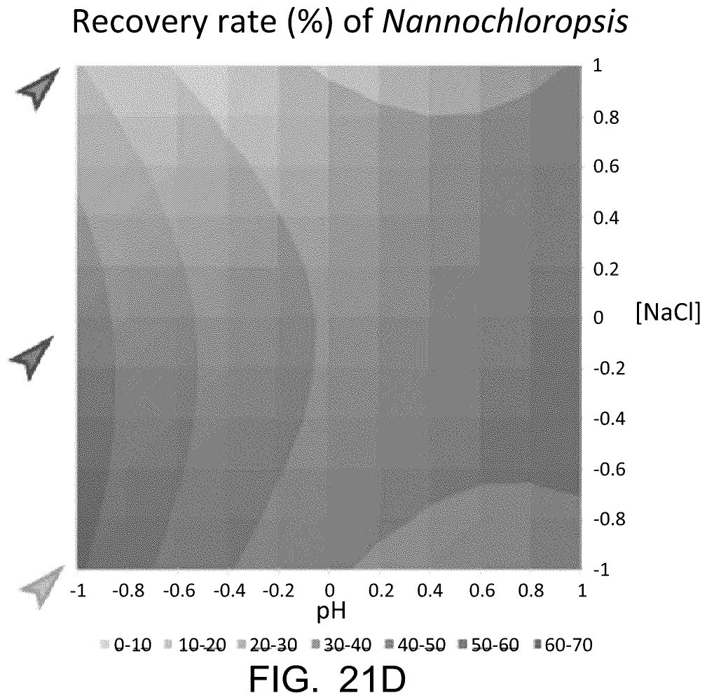

As previously described, the response surfaces were plotted according to the model described in different rotations in order to facilitate visualization and interpretation (FIG. 21A-D). In FIGS. 21A, 21B, and 21C, the response surface is projected in three dimensions with pH values fixed at −1, 0 and 1 levels corresponding to pH values of 3.1, 4.8 and 6.4, respectively. NaCl concentration values were fixed at −1, 0 and 1 levels, which correspond to 0, 20 and 40 g/L, respectively. After calculating the coefficients, the following model was predicted for Nannochloropsis sp.:

y=39.12-14.09x₁−1.19x₂+4.11x₁x₂+11.59x₁²−8.92x₂²

On the response surfaces plotted according to the model described in different rotations in order to facilitate visualization and interpretation, it was possible to observe the optimal point (FIG. 2; orange arrowhead), point of the culture condition (FIG. 21A-D; red arrowhead) and intermediate point for pH and salinity adjustment (FIG. 21A-D; blue arrowhead). The optimum point, as previously described herein, was reached at pH 3.1 and without adding NaCl to the culture medium (FIG. 2; orange arrowhead) corresponding to a recovery of 59.52%. The culture condition point (FIG. 2; red arrowhead), i.e., NaCl at 40 g/L, obtained a recovery of 52.08%, corresponding to an insignificant drop when considering the process conditions. In addition, the intermediate point of the salinity of the culture (FIG. 21A-D; red arrowhead), i.e., NaCl at 20 g/L, obtained a recovery of 64.93%, corresponding to a slightly higher increase than expected for the optimal point, previously described. Therefore, it was again possible to observe that there was cell recovery in the conditions closest possible to the collection step in tanks and raceways for microalgae culture, adding the steps of acidification of the medium and collection and magnetic separation by adding NMOBs.

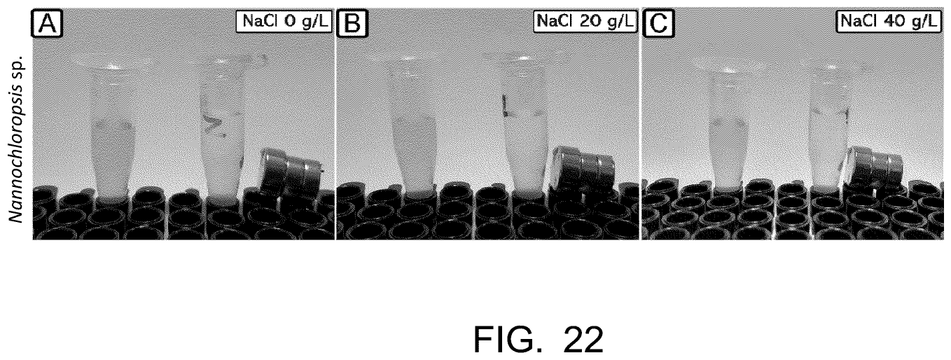

Finally, after collecting the data to generate the response surface, the magnetic concentration of the material was recorded (FIG. 22). A neodymium-boron magnet was positioned and the microalgae biomass flocculated by the NMOBs was concentrated on the side of the tube in response to the generated magnetic field. It was possible to observe the response to the field in all proposed salinity conditions.

After carrying out the experimental planning and the challenge of NMOBs in face of synthetic magnetite nanoparticles and the flocculant, it was possible to observe that the NMOBs described in the present work have the ability to magnetically collect and act in the magnetic separation of microalgae species in hypersaline conditions. The use of nanoparticles in the challenge showed they are inefficient in the cell collection process under high salinity conditions, as well as the chemical flocculant. The visual observation of magnetic collection as shown in FIG. 22 for NMOBs was not seen in the synthetic nanoparticle challenge.

### BIBLIOGRAPHIC REFERENCES

Ike, I. A., & Duke, M. (2018). Synthetic magnetite, maghemite, and hematite activation of persulphate for orange G degradation. Journal of contaminant hydrology, 215, 73-85.
Lam G. P., Zegeye E. K., Vermuë M. H., Kleinegris D. M. M., Eppink M. H. M., Wijffels R. H., Olivieri G. 2015. Dosage effect of cationic polymers on the flocculation efficiency of the marine microalga Neochloris oleoabundans, Bioresour. Technol. 198: 797-802.
Lei, C., Zhang, L., Yang, K. et al. (2016) Toxicity of iron-based nanoparticles to green algae: effects of particle size, crystal phase, oxidation state and environmental aging. Environ Pollut 218, 505-512.
Leon-Reyes, A., Epifani, M., Chávez-Capilla, T., Palma, J., & Díaz, R. (2014). Analysis of the different mechanisms of electrochemical energy storage in magnetite nanoparticles. Int. J. Electrochem. Sci, 9(7), 3837-3845.
Roselet, F., Vandamme, D., Roselet, M. et al. 2017. Effects of pH, Salinity, Biomass Concentration, and Algal Organic Matter on Flocculant Efficiency of Synthetic Versus Natural Polymers for Harvesting Microalgae Biomass. Bioenerg. Res. 10, 427-437 (2017).
Santos, E. C. D. S., Watanabe, A., Vargas, M. D., Tanaka, M. N., Garcia, F., & Ronconi, C. M. (2018). AMF-responsive doxorubicin loaded β-cyclodextrin-decorated superparamagnetic nanoparticles. New Journal of Chemistry, 42 (1), 671-680.
Yoshino, T., Hirabe, H., Takahashi, M., Kuhara, M., Takeyama, H., & Matsunaga, T. (2008). Magnetic cell separation using nano-sized bacterial magnetic particles with reconstructed magnetosome membrane. Biotechnology and bioengineering, 101(3), 470-477.
Vandamme, D., Foubert, I., Meesschaert, B., Muylaert, K. 2010. Flocculation of microalgae using cationic starch. J. Appl. Phycol. 22, 525-530.

1. (canceled)

2. (canceled)

3. (canceled)

4. (canceled)

5. A process for the recovery of microalgae in a suspension, the process comprising:
protonating a cell surface of the microalgae;
adding magnetic nanoparticles of bacterial origin to the suspension; and
applying an external magnetic charge, thus promoting the flocculation of the microalgae.

6. The process of claim 5, wherein the microalgae suspension is selected from a group consisting of: a microalgae culture, a source of salt water, a source of fresh water or an effluent.

7. The process of claim 5, wherein the suspension has a salt concentration equal to or greater than 5 g/mL.

8. The process according to claim 5, wherein protonating the cell surface of the microalgae comprises adding an acidic buffer to the suspension.

9. The process of claim 5, further comprising adding magnetic nanoparticles of bacterial origin at a concentration between 50 and 100 μg/mL to the suspension.

10. The process according to of claim 5, wherein the microalgae is selected from a group consisting of: Chlorella sp., Crypthecodinium sp., Cylindrotheca sp., Desmodesmus sp., Dunaliella sp., Isochrysis sp., Monoraphidium sp., Nannochloris sp., Nannochloropsis, Neochloris sp., Nitzschia sp., Phaeodactylum sp., Porphyridium sp., Scenedesmus sp., Schizochytrium sp., or Tetraselmis sp.

11. A microalgae suspension comprising:
a source selected from a group consisting of a microalgae culture, salt water source, fresh water source, or an effluent;
a salt concentration equal to or greater than 5 g/mL; and
a microalgae selected from a group consisting of: Chlorella sp., Crypthecodinium sp., Cylindrotheca sp., Desmodesmus sp., Dunaliella sp., Isochrysis sp., Monoraphidium sp., Nannochloris sp., Nannochloropsis, Neochloris sp., Nitzschia sp., Phaeodactylum sp., Porphyridium sp., Scenedesmus sp., Schizochytrium sp., or Tetraselmis sp.

12. The microalgae suspension of claim 11, further comprising magnetic nanoparticles of bacterial origin.

13. The microalgae suspension of claim 12, wherein the magnetic nanoparticles of bacterial origin comprise a concentration between 50 and 100 μg/mL.

14. The microalgae suspension of claim 11, further comprising an acidic buffer.
